(19) United States
(12) Patent Application Publication
(10) Pub. No.: US 2025/0329016 A1
(43) Pub. Date: Oct. 23, 2025

(54) DIGITAL IMAGE ANALYSIS

(71) Applicant: Hoffmann-La Roche Inc., Little Falls, NJ (US)

(72) Inventors: Thomas ALBRECHT, Riehen (CH); Josep Arús Pous, Sabadell (ES)

(73) Assignee: Hoffmann-La Roche Inc., Little Falls, NJ (US)

(21) Appl. No.: 19/221,940

(22) Filed: May 29, 2025

Related U.S. Application Data
(63) Continuation of application No. PCT/EP2023/083242, filed on Nov. 28, 2023.

(30) Foreign Application Priority Data
Nov. 30, 2022 (EP) 22210482.0

(51) Int. Cl. G06T 7/00; G06N 20/20; G06T 3/40; G06T 7/194; G06V 10/44; G16H 30/20; G16H 30/40

(52) U.S. Cl. CPC G06T 7/0012; G06N 20/20; G06T 3/40; G06T 7/194; G06V 10/44; G16H 30/20; G16H 30/40; G06T 2207/30096; G06V 2201/07

(57) ABSTRACT

The present invention relates to systems, methods and products for analyzing digital images, in particular digital pathology images.

### FIELD OF INVENTION

The present invention relates to systems, methods and products for analyzing digital images, in particular digital pathology images.

### BACKGROUND TO THE INVENTION

Digital image analysis consists in extracting information from digital images by means of computational techniques. In particular, the availability of rapidly growing datasets of digital images has been key to the success of Machine Learning (ML) algorithms for image processing and analysis.

Digital pathology leverages state-of-the-art ML algorithms to analyze digitized versions of Whole Slide Images (WSI). In this context, WSI are digital images of sample slides (typically glass slides), for example slides stained with haematoxylin and eosin (H&E) or other stains. Samples such as tissue sections can be analyzed through such algorithms, for example to identify histopathological changes or formulate diagnosis. Such algorithms are typically trained on large datasets of manually annotated pathology images.

It is generally believed that the training of ML algorithms for digital image analysis benefits from the availability of larger datasets. Indeed, the amount of training data available may impact the performance of the trained algorithm in detecting and predicting features of the images. However, the training of ML models on large datasets can be demanding in terms of time and computing resources. Moreover, the choice of a particular dataset to train the model can influence the resulting performance of the model.

Therefore there is a need for improved systems and methods to train ML models for digital image analysis and to compare the model performance across training datasets. STATEMENTS OF INVENTION

The present invention relates to systems, methods and products for analyzing digital images, in particular digital pathology images. These methods can be used, among other applications, to detect and reconstruct features in the images, such as for example organs and/or lesions, to determine the degree of disease progression, to assess the response of patients to a treatment in clinical studies, to predict the presence or the likelihood of a lesion.

Thus, according to a first aspect, this invention provides a computer-implemented method of obtaining one or more trained ML models for digital pathology image analysis, the method comprising the steps of: receiving one or more digital pathology images; preprocessing the one or more digital pathology images, wherein preprocessing comprises, for each of the one or more digital pathology images: extracting a plurality of tiles, obtaining a rasterized representation of the extracted tiles, associating, with each tile, tile metadata comprising one or more tiling parameters, and storing a single file, the single file comprising the rasterized tiles and the tiles metadata; obtaining a training dataset comprising the one or more single files stored for the one or more preprocessed digital pathology images; filtering the training dataset using the tiles metadata; training the one or more ML models using the filtered training dataset to obtain one or more trained ML model. The method can further comprise the step of outputting the one or more trained ML models. The step of receiving one or more digital pathology images can comprise receiving annotations associated with the one or more digital pathology images. In such embodiments, the tile metadata can comprise the received annotations associated with the digital pathology image from which the tile was extracted. The method can further comprise obtaining one or more masks associated with the rasterized representation of the extracted tiles. In such embodiments, the tile metadata associated with each tile can comprise one or more masks parameters. The single file can comprise the obtained mask(s). The step of extracting a plurality of tiles for a digital pathology image can comprise obtaining at least a first plurality of tiles associated with a first magnification level and a second plurality of tiles associated with a second magnification level, wherein the tiling parameters associated with the extracted tiles comprise the magnification level used to extract the tiles. The step of extracting a plurality of tiles for a digital pathology image can comprise obtaining at least a first plurality of tiles associated with a first color channel and a second plurality of tiles associated with a second color channel, wherein the tiling parameters associated with the extracted tiles comprise the color channel used to extract the tiles. The step of obtaining one or more masks associated with the rasterized representation of the extracted tiles can comprise analyzing a rasterized representation of the one or more digital pathology images and/or analyzing the rasterized representations of the plurality of extracted tiles, and associating the one or more masks with the corresponding rasterized representations of extracted tiles. Analyzing a rasterized representation of a digital pathology image or tile extracted from a digital pathology image to obtain one or more masks can comprise using one or more algorithms configured to detect the location of one or more features of interest in the digital pathology image or tile. A mask can comprise information indicating whether one or more features of interest are present at a plurality of locations of a digital pathology image or tile. Features of interest can comprise eg foreground/background, individual cells, groups of cells, cell boundaries, organs, cells with a predetermined phenotype (eg cell type, positive for a predetermined marker associated with a signal in the image, etc).

The present inventors have identified that by storing the information of the preprocessing step in a single container file it is possible to reuse the file to train different ML models or the same ML model multiple times without the need to reprocess the raw data every time. For example, new images can be preprocessed and corresponding container files added to a training dataset used to train the model or models on a larger training dataset without the need to reprocess the pre-existing images.

The present inventors have also identified that by storing the information of the preprocessing step in a single container file it is possible to have direct access to the preprocessed images, without any additional preparation. This includes the possibility to access any subset of preprocessed images contained in the container file, for example a random subset of the training dataset or a subset based on the tile metadata, for example a subset of images tiled with a given magnification level. This allows to compare the performance of a single ML model trained on different subsets, for example, a single ML model trained on images tiled with different magnification levels. Additionally, this allows to divide the training dataset in batches that can be used to train different ML models in parallel.

The present inventors have also identified that by storing the information of the preprocessing step in a single container file it is possible to standardize the format of the preprocessed images, thus allowing for a training dataset of comparable items.

The present inventors have also identified that by storing the information of the preprocessing step in a single container file it is easier to share and distribute the data among different models and projects.

The method may have one or more of the following features.

The step of receiving one or more digital pathology images can comprise receiving one or more digital pathology images from a user (e.g. through a user interface), from a computer, imaging device or data store. The method can further comprise acquiring the digital pathology images from at least one sample (e.g. by means of a digital microscope). The sample can have been previously obtained from a patient. The patient can be a human patient. The patient can be an adult patient. The patient can be a paediatric patient. The patient can be a model animal. The patient can be a mammalian. The patient can be a healthy patient. The patient can be a patient that has been diagnosed as having a disease or disorder or being likely to have a disease or disorder. The digital pathology images can be digitized versions of images of glass slides on which tissue samples (eg sections from tissue blocks) or cells samples are supported. The digital pathology images can be digitized versions of images of samples obtained from a single patient or a plurality of patients. The digital pathology images can be digitized versions of glass images of tissues, wherein the tissues can be collected at the same point in time or at different points in time.

The one or more tiling parameters can comprise one or more parameters associated with the step of extracting the plurality of tiles (such as e.g. tiles coordinates, magnification level (resolution), color channel, etc.). Tiles coordinates characterize the location of a tile in the digital pathology image from which the tile is extracted. Magnification levels (resolutions) can comprise for example 1×, 2×, 5×, 10×, 20×. The magnification level can define the number of tiles extracted from the digital pathology image. Color channels can comprise for example red, green, blue, RGB, hue, saturation lightness, HLS.

The tiling parameters associated with a tile can comprise the number of pixels in the tile. The step of obtaining a rasterized representation can comprise obtaining one or more values for each of a plurality of pixels in an image, wherein the one or more values quantify color and/or tonal information at the location of the respective pixel. The step of obtaining a rasterized representation can comprise for example converting from vector representation to rasterized representation.

Optionally, the method can further comprise the step of updating the tiling parameters to include the number of pixels resulting from the step of obtaining the rasterized representation of a respective tile.

The optional step of obtaining masks associated with the extracted rasterized tiles can comprise executing one or more image analysis algorithms, for example a mask CNN, a transformer-based algorithm or an attention-based algorithm. The obtained masks can comprise a foreground/background mask. A foreground/background mask can comprise for each pixel in a rasterized image a first value for pixels identified as foreground and a second value for pixels identified as background. A foreground/background mask can be a tissue/background mask. The masks parameters can comprise one or more parameters each associated with the presence of a feature of interest (eg structure or signal) in the extracted tile, for example the percentage of tissue in each tile. The obtained masks can comprise organ identification masks, comprising for each pixel in a rasterized image a value for pixels identified as an organ and classified as a certain type of organ. Thus, the optional step of obtaining masks can comprise executing one or more image analysis algorithms configured to identify organs. The obtained masks can comprise organoid identification masks, comprising for each pixel in a rasterized image a value for pixels identified as an organoid and classified as a certain type of organoid. Thus, the optional step of obtaining masks can comprise executing one or more image analysis algorithms configured to identify organoids. The obtained masks can comprise cellular tissue identification masks, comprising for each pixel in a rasterized image a value for pixels identified as a cellular tissue and classified as a certain type of cellular tissues. Thus, the optional step of obtaining masks can comprise executing one or more image analysis algorithms configured to identify cellular tissue. The obtained masks can comprise cell type identification masks, comprising for each pixel in a rasterized image a value for pixels identified as cells and classified as a certain type of cells. Thus, the optional step of obtaining masks can comprise executing one or more image analysis algorithms configured to identify cells. The masks parameters can further comprise one or more parameters associated with the step of identifying organs, organoids, cellular tissues and/or cell types, for example the number and the type of organs and/or organoids present in each tile, the number and the type of cellular tissues present in each tile, and/or the number and the type of cells present in each tile. Optionally, the masks parameters can comprise, for example, masks size (width, height), resolution, coordinates.

The step of storing, for each of the one or more digital pathology images, a single file can comprise storing, for each of the one or more digital pathology images, a single container file, for example a HDF file. In contrast to archive files, such as for example ZIP files, container files allow for random access of the items. The single file stored for each of the one or more digital pathology images can further comprise a reduced-resolution version of each of the one or more digital pathology images. The method can further comprise obtaining a reduced-resolution version of each of the one or more digital pathology images. The method can further comprising displaying to a user, through a user interface, the reduced-resolution version of each of the one or more digital pathology images. This can serve for visualization purposes and can enable users to navigate a digital pathology dataset (eg dataset to be used for training a ML algorithm) more quickly without having to access all of the information in it contained, thus using less computer power to display the images, as well as allow for a quick selection of images in the training dataset of interest for any specific purpose.

The step of filtering the training dataset using the tiles metadata can comprise for example selecting one or more single files and/or a plurality of tiles from each of one or more single files from the training dataset that satisfy one or more predetermined criteria that apply to one or more parameters of the tiles metadata. In embodiments, the one or more predetermined criteria apply to the following parameters and combinations thereof: tile coordinates; magnification levels (resolutions); color channels; number of pixels resulting from the step of obtaining the rasterized representation of a respective tile; masks size (width, height), masks resolution (dependent on or independent of magnification levels), masks coordinates; percentage of tissue over background; number and type of organs; number and type of organoids; number and type of cellular tissues; number and type of cells; received annotations.

The step of training the one or more ML models using the filtered training dataset to obtain one or more trained ML models can comprise training a single ML model using the filtered training dataset, training a single ML model using a plurality of subsets of the filtered training dataset in series, training a single ML model using a plurality of subsets of the filtered training dataset in parallel, training of multiple ML models in series using the filtered training dataset, training multiple ML models in parallel using the filtered training dataset, training multiple ML models using respective subsets of the filtered training dataset in series, training multiple ML models using respective subsets of the filtered training dataset in parallel. The step of training the one or more ML models can further comprise evaluating the training performance of the one or more trained ML models. In an embodiment, the training performance is evaluated in terms of computing time and/or computing resources and/or prediction accuracy. The filtered dataset can also be used to test one or more ML models trained according to methods herein, for example in the following ways: testing a single ML model using the filtered dataset, testing a single ML model using a plurality of subsets of the filtered dataset in series, testing a single ML model using a plurality of subsets of the filtered dataset in parallel, testing multiple ML models in series using the filtered dataset, testing multiple ML models in parallel using the filtered dataset, testing multiple ML models using respective subsets of the filtered dataset in series, testing multiple ML models using respective subsets of the filtered dataset in parallel. The step of testing the one or more trained ML models can further comprise evaluating the testing performance of the one or more tested ML models. In an embodiment, the testing performance is evaluated in terms of computing time and/or computing resources and/or prediction accuracy.

According to a second aspect, there is provided a method of selecting one or more ML models for digital pathology image analysis from one or more trained ML models obtained according to the first aspect, the method comprising the steps of: receiving a test dataset comprising at least one digital pathology image; testing the one or more trained ML models using the test dataset; calculating one or more evaluation metrics for each of the one or more tested ML models; and selecting one or more of the tested ML models using one or more predetermined criteria applying to the one or more evaluation metrics. The method can comprise ranking the one or more tested ML models using the calculated evaluation metric(s) for each of the one or more tested ML models; and selecting one or more ML models can comprise selecting the one or more highest-ranked tested ML models. The evaluation metric(s) can be selected from for example: accuracy, precision, recall, specificity, and confusion matrix (e.g. number of false positives and false negatives). The step of selecting one or more of the tested ML models using one or more predetermined criteria applying to the one or more evaluation metrics can comprise selecting one or more of the tested ML models using one or more predetermined criteria that apply to a performance evaluated according to an embodiment of the first aspect and/or to one or more evaluation metrics according to the present aspect. The step of ranking the one or more tested ML models can further comprise using a combination of the calculated evaluation metric and the evaluated training performance according to the first aspect. Methods according to the present aspect can comprise performing the steps of any embodiment of the first aspect.

According to a third aspect, there is provided a method of using a trained ML model, obtained according to the first aspect, to analyze digital pathology images, the method comprising the steps of: receiving at least one digital pathology image; extracting, via the trained ML model, features of the received at least one digital pathology image, wherein extracting features comprises detecting and/or predicting the presence of objects, structures, and/or lesions in the received at least one digital pathology image. The ML model can be a ML model configured to detect the presence of lesions in organs and optionally determined one or more properties of the detected lesions (e.g. size, shape), to determine the degree of disease progression, to assess the response of patients to a treatment in clinical studies, to predict the presence or the likelihood of a lesion. The method according to the present aspect can include the steps of any embodiment of the first or second aspects. For example, the methods of the present aspect can comprise the steps of obtaining the trained ML models for digital pathology image analysis using a method according to the first aspect. The methods according to the present aspect can comprise the steps of obtaining a processed digital pathology image dataset from the received at least one digital pathology image according to any embodiment of the fourth aspect.

According to a fourth aspect, there is provided a method of obtaining a processed digital pathology image dataset, the method comprising: receiving one or more digital pathology images; preprocessing the one or more digital pathology images, wherein preprocessing comprises, for each of the one or more digital pathology images, extracting a plurality of tiles, obtaining a rasterized representation of the extracted tiles, associating, with each tile, tile metadata comprising one or more tiling parameters, and storing a single file, the single file comprising the rasterized tiles and the tiles metadata; obtaining a processed digital pathology image dataset comprising the one or more single files stored for the one or more preprocessed digital pathology images. The step of receiving one or more digital pathology images can comprise receiving annotations associated with the one or more digital pathology images. In such embodiments, the tile metadata can comprise the received annotations associated with the digital pathology image from which the tile was extracted. The method can further comprise obtaining one or more masks associated with the rasterized representation of the extracted tiles. In such embodiments, the tile metadata associated with each tile can comprise one or more masks parameters. The single file can comprise the obtained mask(s). The methods according to the present aspect can have any of the features described in relation to the first aspect.

According to a fifth aspect, there is provided a method of using a processed digital pathology image dataset obtained according to the fourth aspect to train one or more ML models, the method comprising: filtering the dataset using the tiles metadata; training the one or more ML models using the filtered dataset.

According to a sixth aspect, there is provided a method of using a processed digital pathology image dataset obtained according to the fourth aspect to test one or more ML models trained according to the first aspect, the method comprising: filtering the dataset using the tiles metadata; testing the one or more ML models using the filtered dataset.

According to a further aspect, there is provided a system comprising a processor, and a computer readable medium comprising instructions that, when executed by the processor, cause the processor to perform the computer-implemented steps of the method of any preceding aspect. The system can further comprise means for acquiring digital pathology images from a sample, for example a digital microscope. According to a further aspect, there is provided a non-transitory computer readable medium or media comprising instructions that, when executed by at least one processor, cause the at least one processor to perform the method of any embodiment of any aspect described herein. According to a further aspect, there is provided a computer program comprising code which, when the code is executed on a computer, causes the computer to perform the method of any embodiment of any aspect described herein.

### BRIEF DESCRIPTION OF THE FIGURES

FIG. 1 illustrates an embodiment of a system that can be used to implement one or more aspects described herein.

FIG. 2 is a flow diagram showing, in schematic form, a method of obtaining one or more trained ML models for digital pathology image analysis, according to the invention.

FIG. 3 is a flow diagram showing, in schematic form, a method of obtaining in series two trained ML models for digital pathology image analysis, according to the invention.

FIG. 4 is a flow diagram showing, in schematic form, a method of obtaining in parallel two trained ML models for digital pathology image analysis, according to the invention.

FIG. 5 is a flow diagram showing, in schematic form, a method of selecting one or more optimal ML models for digital pathology image analysis, according to the invention.

FIG. 6 is a flow diagram showing, in schematic form, a method of using a trained ML model to analyze digital pathology images, according to the invention.

FIG. 7 is a flow diagram showing, in schematic form, a method of comparing the performance of two trained ML models, according to the invention.

FIG. 8 is a flow diagram showing, in schematic form, a method of selecting a subset of an image dataset, according to the invention.

FIG. 9 is an example of a single file stored during preprocessing of a digital pathology image, according to the invention.

### DETAILED DESCRIPTION

In describing the present invention, the following terms will be employed, and are intended to be defined as indicated below.

As used herein “data” and “images” are used interchangeably unless otherwise specified.

As used herein ML “algorithms” and “models” are used interchangeably unless otherwise specified.

As used herein “training” a ML model assumes the standard meaning known to the person skilled in the art, and comprises finding the best combination of model parameters, e.g. weights and bias (depending on the architecture of the model), to minimize a loss function over training data.

As used herein “features” in the images can comprise objects, structures, lesions, organs, cellular tissues, crypts, tumors, etc. “Detecting features” is used with the meaning of identifying the presence and/or location of said features. “Reconstructing features” is used with the meaning of characterizing said features, for example obtaining their size, shape, contrast. “Predicting features” is used with the meaning of estimating a probability of the presence of said features and/or of their characteristics, e.g. size, shape, contrast.

As used herein “tile metadata” can comprise one or more of “tiling parameters”, “masks parameters”, “annotations.” “Tiling parameters” are parameters associated with the tiling process, for example tiles coordinates, magnification levels (resolutions), color channels. “Masks parameters” are parameters associated with the process of obtaining the masks, for example one or more parameters each associated with the presence of a structure or signal in the extracted tile, for example the percentage of tissue in each tile, one or more parameters associated with the step of identifying organs, for example the number and the type of organs, the number and the type of cellular tissues, the number and the type of organoids present in each tile.

The systems and methods described herein can be implemented in a computer system, in addition to the structural components and user interactions described. As used herein, the term “computer system” includes the hardware, software and data storage devices for embodying a system and carrying out a method according to the described embodiments. For example, a computer system can comprise one or more central processing units (CPU) and/or graphics processing units (GPU), input means, output means and data storage, which can be embodied as one or more connected computing devices. Preferably the computer system has a display or comprises a computing device that has a display to provide a visual output display. The data storage can comprise RAM, disk drives, solid-state disks or other computer readable media. The computer system can comprise a plurality of computing devices connected by a network and able to communicate with each other over that network. It is explicitly envisaged that computer system can consist of or comprise a cloud computer.

The methods described herein are computer implemented unless context indicates otherwise. Indeed, the training, testing and use of the algorithms for digital pathology image analysis are such that the methods described herein are far beyond the capability of the human brain and can not be performed as a mental act. The methods described herein can be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media, magnetic tape; optical storage media such as optical discs or CD-ROMs; electrical storage media such as memory, including RAM, ROM and flash memory; hybrids and combinations of the above such as magnetic/optical storage media.

### Systems

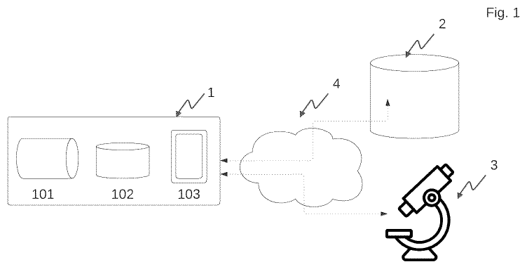

FIG. 1 illustrates and embodiment of a system that can be used to implement one or more aspects described herein. The system comprises a computing device 1, which comprises a processor 101 and a computer readable memory 102. In the embodiment shown, the computing device 1 also comprises a user interface 103, which is illustrated as a screen but can include any other means of conveying information to a user such as e.g. through audible or visual signals. The computing device 1 is communicably connected, such as e.g. through a network, to digital pathology images acquisition means, such as a digital microscope, and/or to one or more databases 2 storing digital pathology images. The one or more databases 2 can further store one or more of: control data, parameters (such as e.g. thresholds derived from control data, parameters used for normalization, etc.), clinical and/or patient related information, etc. The computing device can be a smartphone, tablet, personal computer or other computing device. The computing device can be configured to implement a method of obtaining one or more trained ML models for digital pathology image analysis, as described herein. In alternative embodiments, the computing device 1 is configured to communicate with a remote computing device (not shown), which is itself configured to implement a method of obtaining one or more trained ML models for digital pathology image analysis, as described herein. In such cases, the remote computing device can also be configured to send the result of the method of obtaining one or more trained ML models for digital pathology image analysis. Communication between the computing device 1 and the remote computing device can be through a wired or wireless connection, and can occur over a local or public network 4 such as e.g. over the public internet. The digital pathology image acquisition means 3 can be in wired connection with the computing device 1, or can be able to communicate through a wireless connection, such as e.g. through WiFi and/or over the public internet, as illustrated. The connection between the computing device 1 and the digital pathology image acquisition means 3 can be direct or indirect (such as e.g. through a remote computer). The digital pathology image acquisition means 3 are configured to acquired digital pathology images from a subject, for example digitized WSI of tissue samples. In some embodiments, the digital pathology images can have been subject to one or more preprocessing steps (eg cropping, resizing, normalizing, etc) prior to performing the methods described herein.

### Methods

In embodiments of the present invention, one or more trained ML models for digital pathology image analysis are obtained. In these embodiments, the models are obtained by a computer-implemented method or tool that takes as input one or more digital pathology images from at least one patient, and produces as output one or more ML models trained using the inputted digital pathology image or images. The present inventors have demonstrated that by preprocessing the one or more digital pathology images in such a way as to obtain a single file for each digital pathology image as described herein, the single file comprising rasterized tiles of the image, tiles metadata and optionally annotations received with the image and optionally a reduced-resolution version of the image, it is possible to train one or more ML models using a training dataset comprising the one or more single files obtained for each digital pathology image. Training the one or more ML models using a training dataset comprising such one or more single files has the advantage of the flexibility of filtering the dataset using the tiles metadata contained in the single files and training the one or more ML models using the filtered dataset. This allows one to train one or more ML models with differently filtered datasets from each single file, without the need to reprocess the raw data. This also allows one to access the items in each single file in a random way. This also allows one to standardize the format of the preprocessed images within each single file and among different single files. This also allows one to share and distribute the training datasets more easily.

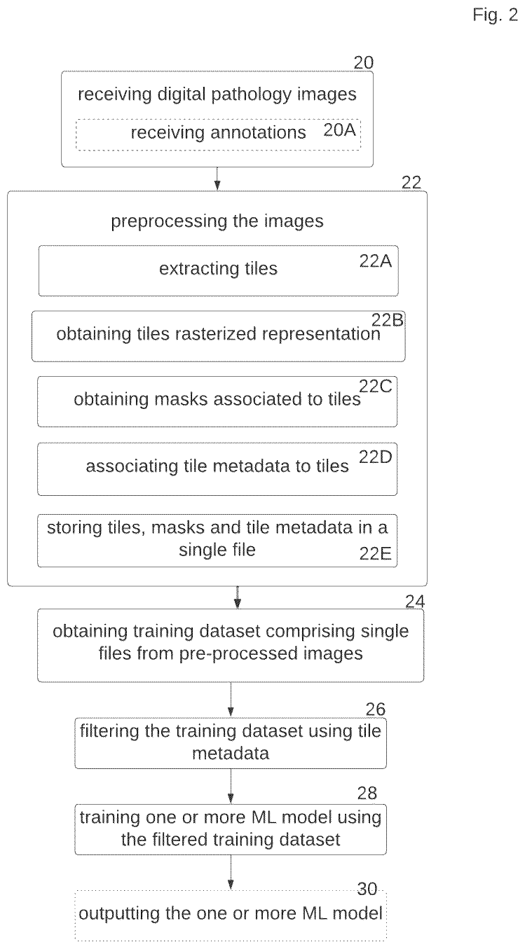

FIG. 2 is a flow diagram showing, in schematic form, a method of obtaining one or more trained ML models for digital pathology image analysis, according to the invention. At step 20, one or more digital pathology images are received. This can comprise optionally receiving annotations associated to the one or more digital pathology images (20A), for example ground-truth annotations indicating the presence of a lesion and/or an organ. Such annotations can be received in the form of a written text, a mark or an overlay on the image, as tabular data or in any other suitable way. At step 22 the digital pathology images can be preprocessed. Preprocessing can comprise several steps performed for each digital pathology image. At step 22A, a plurality of tiles can be extracted from each digital pathology image. This can be done with a tiling algorithm that slices the image into a grid, each cell of the grid being a tile. The tiling algorithm can be configured by user-defined tiling parameters, such as e.g. tiles size or tiles coordinates, magnification levels (resolutions), color channels. Step 22A can comprise obtaining at least a first plurality of tiles associated with a first magnification level and a second plurality of tiles associated with a second magnification level. Examples of magnification levels used are 1×, 2×, 5×, 10×, 20×. At step 22B, a rasterized representation of the extracted tiles is obtained. For example, each tile can be represented as a matrix, of which rows and columns correspond to pixels and the matrix values correspond to pixel intensities. Each tile can be represented as several matrices, for example one per color channel (e.g. red, green, blue). Step 22B can optionally further comprise the step of updating the tiling parameters to include the number of pixels resulting from the rasterized representation of a respective tile. At step 22C, one or more masks associated with the rasterized representation of the extracted tiles are obtained. This step can comprise executing one or more image analysis algorithms, for example a mask CNN, a transformer-based algorithm or an attention-based algorithm. This step can comprise obtaining one or more masks by analyzing the one or more digital pathology images and/or obtaining one or more masks by analyzing the rasterized representations of the plurality of extracted tiles, and associating the one or more masks with the corresponding rasterized representations of extracted tiles. Step 22C comprises obtaining masks parameters associated with the process of obtaining one or more masks. For example, a foreground/background mask can be obtained per tile, in particular a foreground/background mask can be a tissue/background mask and the masks parameters can comprise the percentage of tissue in the tile. Step 22C can comprise obtaining organ identification masks, organoid identification masks, cellular tissue identification masks or cell type masks. The masks parameters can further comprise one or more parameters associated with the step of identifying organs, for example the number and the type of organs and/or organoids present in each tile, the number and the type of cellular tissues present in each tile. Optionally, the masks parameters can comprise, for example, masks size (width, height), resolution, coordinates. At step 22D, tile metadata is associated to each tile, with the tile metadata comprising one or more tiling parameters, one or more of masks parameters, and optionally one or more of the received annotations associated to the digital pathology image from which the tile is extracted. At step 22E, a single file is stored per digital pathology image, the file comprising the rasterized tiles, the obtained masks and the tiles metadata. The single file can be a single container file, in particular a HDF file. Items in the single container file, in particular a HDF file, can be randomly accessed. The single file can further comprise a reduced-resolution version of the digital pathology image.

Step 22E can optionally comprise a step of displaying to a user, through a user interface, the reduced-resolution version of the digital pathology image contained in the single file. At step 24, a training dataset is obtained, the dataset comprising each single file stored per preprocessed digital pathology image. At step 26, the training dataset is filtered using the tiles metadata. This step can comprise for example selecting one or more single files and/or a plurality of tiles from each of one or more single files from the training dataset that satisfy one or more predetermined criteria that apply to one or more parameters of the tiles metadata. For example, within each file tiles can be chosen obtained with a certain magnification level. For example, within each file tiles can be chosen wherein a certain organ is present (eg as indicated in masks parameters). For example, within each file tiles can be chosen with or above or below a predetermined percentage of tissue over background. At step 28, the one or more ML models are trained using the filtered training dataset. This step can comprise training a single ML model using the filtered training dataset, training a single ML model using a plurality of subsets of the filtered training dataset in series, training a single ML model using a plurality of subsets of the filtered training dataset in parallel, training of multiple ML models in series using the filtered training dataset, training multiple ML models in parallel using the filtered training dataset, training multiple ML models using respective subsets of the filtered training dataset in series, training multiple ML models using respective subsets of the filtered training dataset in parallel. This step can further comprise evaluating the training performance of the one or more trained ML models. For example, a single ML model can be trained on the training dataset filtered on a magnification level 1× and on a training dataset filtered on a magnification level 2×. The training using the two filtered datasets can be done in parallel and the training performance, for example in terms of computing resources and time, can be evaluated. At optional step 30, the one or more trained ML models can be outputted to the user. Alternatively, the one or more ML models can be stored in a local server or a cloud server. The trained ML models can thus be fetched when needed on a number of projects.

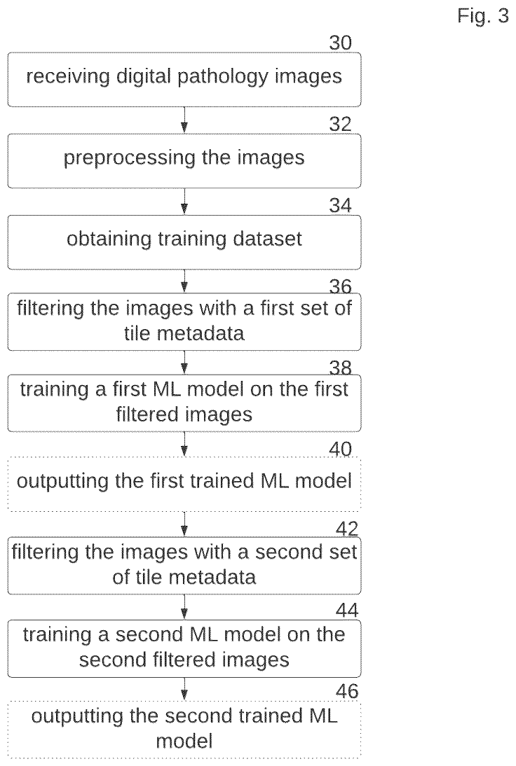

FIG. 3 is a flow diagram showing, in schematic form, a method of obtaining in series two trained ML models for digital pathology image analysis, according to the invention. At steps 30, 32, 34, digital pathology images are received and preprocessed and a training dataset is obtained in a manner as hereinbefore described. At step 36, the obtained training dataset is filtered with a first set of tile metadata. At step 38 a first ML model is trained using the first filtered training dataset.

At optional step 40, the first trained ML model is outputted. At step 42, the obtained training dataset is filtered with a second set of tile metadata. At step 44 a second ML model is trained using the second filtered training dataset. At optional step 46, the second trained ML model is outputted. The two ML models can be trained sequentially on differently filtered training datasets without the need to preprocess the training dataset twice.

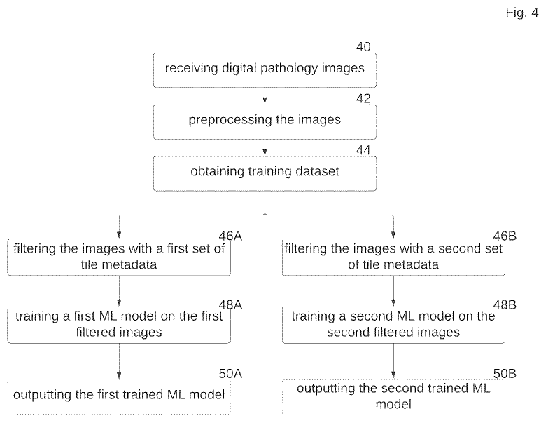

450 FIG. 4 is a flow diagram showing, in schematic form, a method of obtaining in parallel two trained ML models for digital pathology image analysis, according to the invention. At steps 40, 42, 44, digital pathology images are received and preprocessed and a training dataset is obtained in a manner as hereinbefore described. At steps 46A and 46B, a first set of filtered data is obtained by filtering the obtained training dataset using a first set of tile metadata and in parallel a second set of filtered data is obtained by filtering the obtained training dataset. The filtering can use the same or different one or more predetermined criteria that apply to the tile metadata. For example, the first set of filtered data can be obtained by selecting tiles associated with a first magnification level (comprised in the tile metadata) and the second set of filtered data can be obtained by selecting tiles associated with a second magnification level (comprised in the tile metadata). The first and second magnification levels can be different from each other. This may be used for example to compare the performance of models trained on data using different magnification levels. At step 48A and 48B, a first ML model is trained using the first filtered training dataset and in parallel a second ML model is trained using the second filtered training dataset. At optional steps 50A and 50B, the first trained ML model is outputted and in parallel the second trained ML model is outputted. The two ML models can be trained in parallel on differently filtered training datasets without the need to preprocess the training dataset twice.

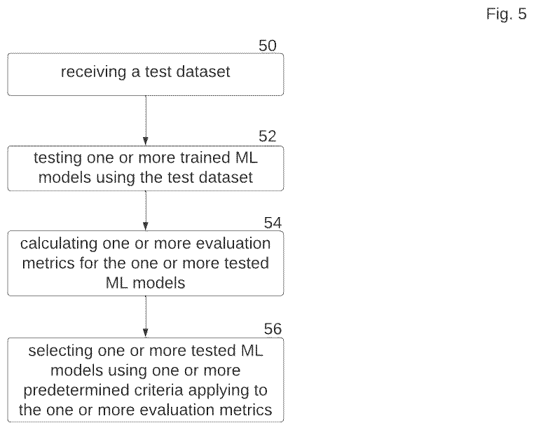

FIG. 5 is a flow diagram showing, in schematic form, a method of selecting one or more ML models for digital pathology image analysis, according to the invention. The models can be considered optimal according to one or more predetermined criteria. At step 50, a test dataset is received. The test dataset can be for example a digital pathology image dataset, in particular a digital pathology image dataset obtained in a manner according to the invention. For instance, the test dataset can comprise single files stored from the digital pathology images in a manner according to the invention. At step 52, one or more ML models trained according to the invention as hereinbefore described are tested using the test dataset. At step 54, for the one or more tested ML models one or more evaluation metrics are calculated. The evaluation metrics can comprise for example: accuracy, precision, recall, specificity, and confusion matrix (e.g. number of false positives and false negatives). At step 56, one or more tested ML models are selected using one or more predetermined criteria applying to the one or more evaluation metrics. This step can further comprise using as predetermined criteria a combination of the calculated evaluation metrics and the evaluated training or testing performance according to the first aspect. This step can further comprise ranking the one or more tested ML models using the one or more predetermined criteria applying to the one or more evaluation metrics, and selecting the one or more tested ML models with highest rank or ranks. This step can further comprise ranking the one or more tested ML models using as predetermined criteria a combination of the calculated 485 evaluation metrics and the evaluated training or testing performance according to the first aspect.

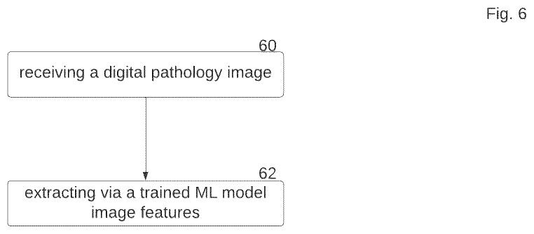

FIG. 6 is a flow diagram showing, in schematic form, a method of using a trained ML model to analyze digital pathology images, according to the invention. At step 60, a digital pathology image is received. At step 62, a trained ML model according to the invention as hereinbefore described is used to extract features from the digital pathology image. The ML model can be a supervised model, for example a Deep Neural Network (DNN), a Convolutional Neural Network (CNN), a Region-based CNN (RCNN). In this step, extracting features can comprise detecting and/or predicting the presence of objects, structures, lesions in the received digital pathology image.

All digital pathology images obtained can be preprocessed as described in relation to FIG. 2, using default image analysis algorithms and any set of tiling parameters and then stored in a database. Any subsequently received image can be preprocessed in the same way. ML models can then be trained as described in relation to FIG. 2, FIG. 3 or FIG. 4, at any point (using any subset or augmented set of data) using the same process without having to re-adapt the training process or reprocess the images to be used every time. Trained ML models can then be used on any new image that is added to the data set and preprocessed in the same way as hereinbefore described, or on any subset of the preprocessed data, as described in relation to FIG. 6.

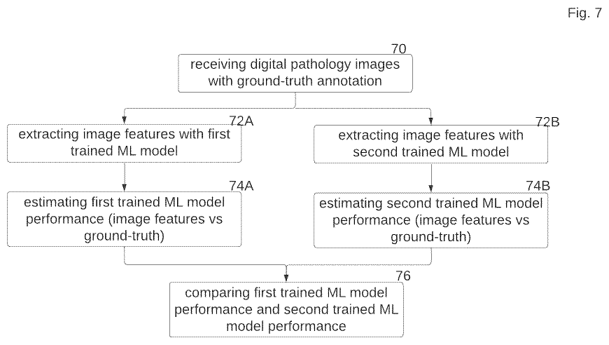

FIG. 7 is a flow diagram showing, in schematic form, a method of comparing the performance of two trained ML models, according to the invention. At step 70, digital pathology images with ground-truth annotations are received. At steps 72A and 72B, image features are extracted from the digital pathology images with a first trained ML model and a second trained ML model in parallel. The two ML models can be supervised models, for example Deep Neural Networks (DNNs), Convolutional Neural Networks (CNNs), Region-based CNNs (RCNNs). At steps 74A and 74B, the performance of the first trained ML model and the performance of the second trained ML model is evaluated in parallel, based on the difference between the ground-truth and the extracted image features with the first trained ML model and with the second ML model respectively. The difference can be estimated in terms of a loss function, for example a regression loss function, a mean absolute error loss function, a cross-entropy loss function. Steps 72B and 74B can also be executed in sequence after steps 72A and 72B. At step 76, the calculated performance of the first trained ML model is compared with the calculated performance of the 515 second trained ML model.

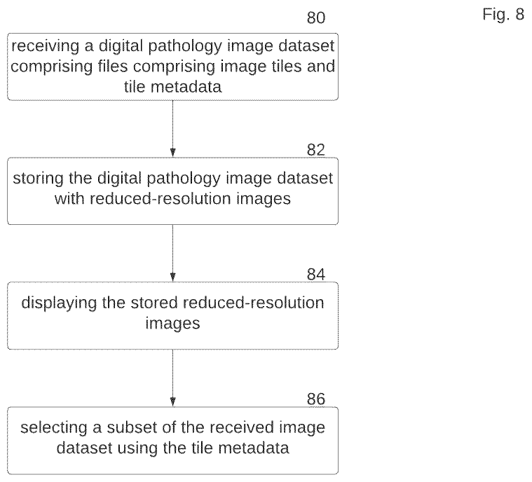

FIG. 8 is a flow diagram showing, in schematic form, a method of selecting a subset of an image dataset, according to the invention. At step 80, a digital pathology image according to the invention as hereinbefore described is received. In particular, the digital pathology image dataset can comprise single files obtained from the digital pathology images, the single files comprising tiles and tile metadata. At step 82, reduced-resolution versions of the digital pathology images in the received dataset are stored, for example as thumbnails in the single files obtained from each of the digital pathology images. At step 84, reduced-resolution versions of the digital pathology images are displayed to the user. This step allows the user to navigate quickly through the images contained in the dataset without having to display their high-resolution version or their tiles, and facilitates the user in selecting a subset of the received digital pathology image dataset. At step 86, a subset of the received digital pathology image dataset is selected using the tile metadata. For example subsets can be selected based on tiles extracted with a certain magnification level, or based on tiles wherein a certain organ is present, or based on tiles wherein a certain percentage of tissue over background is present.

The following is presented by way of example and is not to be construed as a limitation to the scope of the claims.

### EXAMPLES

The examples below illustrate the utility of the methods of the present invention in the particular context of training ML models for digital pathology image analysis. In particular, Example 1 shows a possible composition of a single file used for training a ML model as described herein. Example 2 shows the steps to create such a file. Example 3 shows its possible usage for digital pathology image analysis.

### Example 1—Composition of an Oven Ready Dataset (ORD)

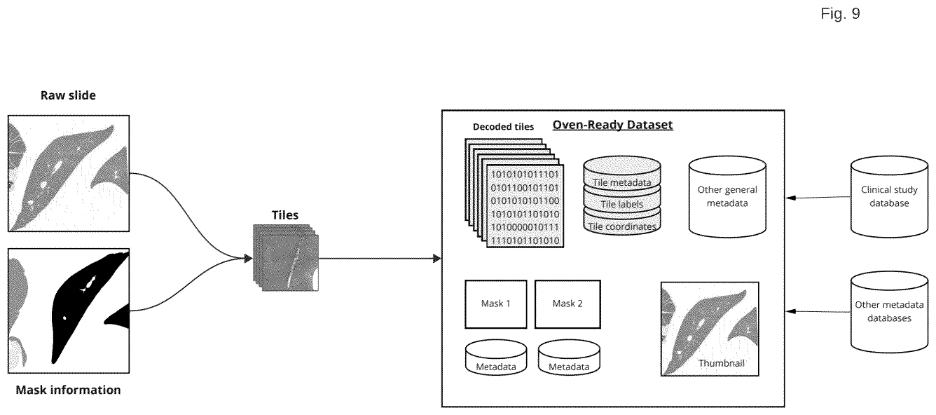

ORDs are single files that contain all the information obtained from a WSI extracted at one or more given magnification levels (e.g. 1×, 2×, 5×, 10×, 20×). ORDs can also contain overlays of the images, called masks, that label the content in the WSI. ORDs can also contain masks-associated metadata, as well as general metadata (e.g. clinical safety assessments, lab annotations). ORDs contain pre-processed WSI data that can be reused in different projects without the need to reprocess the raw data every time. ORDs allows for direct access of the pre-processed data, without any additional preparation. The composition of an ORD can vary depending on the WSI and the available information. FIG. 9 shows an example of an ORD, containing:
A list of the extracted tiles (decoded tiles) at one or more given magnification levels, represented in a raw binary format (e.g. three matrices with R, G, B colors);
Tile metadata, tile labels, tiles coordinates;
Other general metadata (e.g. clinical safety assessments, lab annotations);
A thumbnail of the whole image at reduced resolution, useful for visualization purposes;
A set of masks, e.g. foreground/background masks, organ identification masks, etc. Depending on the use case, there can be masks containing additional information from the WSI. Each mask comes with masks metadata that allow one to position the mask over the WSI, for example masks width, masks height, masks resolution.

### Example 2—Creation of an ORD

In this example, an ORD is created with the following steps:
Raw WSI are obtained from a data source (e.g. Arvados);
Foreground/background masks are calculated.
Raw tiles are obtained at a given magnification level using a tiling algorithm.
Organ identification masks and optionally additional masks with additional information are obtained. These masks can be stitched together to have a single mask per WSI.
Tile metadata are calculated, e.g. what percentage of tissue is present in the tile, which organs are present in the tile, etc).
Additional metadata are fetched from databases.
The ORD is assembled and stored, for example as a HDF5 file, in a data source (e.g. Arvados).

### Example 3—Usage of the ORD for digital pathology image analysis

A training dataset can be built with ORDs of the images at a given magnification level. Alternatively, a training dataset can be built with ORDs of the images that contain a given organ. Alternatively, a training dataset can be built with ORDs of the images that contain a given percentage of tissue.

ORDs of interest to build the desired datasets are obtained from a data source and any ML model is trained using the desired datasets. The advantages of training ML models on ORDs comprise the fact that all data is stored in one single files, and not a large amount of folder and files; data is in a ready-to-use format, so no normalization or standardization is required; files are compressed, thus smaller than the equivalent multiple files one would need to store the separate information (tiles, metadata, thumbnail, . . . ). ORDs are easily sharable and usable in different projects and models.

### EMBODIMENTS

1. In an embodiment, a computer-implemented method of obtaining one or more trained machine-learning models for digital pathology image analysis is disclosed, the method comprising the steps of:
   a. receiving one or more digital pathology images, optionally comprising receiving annotations associated to the one or more digital pathology images;
   b. preprocessing the one or more digital pathology images, wherein preprocessing comprises, for each of the one or more digital pathology images:
      i. extracting a plurality of tiles;
      ii. obtaining a rasterized representation of the extracted tiles;
      iii. obtaining one or more masks associated with the rasterized representation of the extracted tiles;
      iv. associating, with each tile, tile metadata comprising one or more tiling parameters, one or more masks parameters, and optionally the received annotations associated to the digital pathology image from which the tile is extracted;
      v. storing a single file, the single file comprising the rasterized tiles, the obtained masks and the tiles metadata;
   c. obtaining a training dataset comprising the one or more single files stored for the one or more preprocessed digital pathology images;
   d. filtering the training dataset using the tiles metadata;
   e. training the one or more machine-learning models using the filtered training dataset to obtain one or more trained machine-learning models;
   f. optionally outputting the one or more trained machine-learning models.
2. In an embodiment, the method of embodiment 1 is disclosed, wherein the step of extracting a plurality of tiles can comprise obtaining at least a first plurality of tiles associated with a first magnification level.
3. In an embodiment, the method of any preceding embodiments is disclosed, wherein the step of extracting a plurality of tiles can comprise obtaining at least a first plurality of tiles associated with a first magnification level and a second plurality of tiles associated with a second magnification level.
4. In an embodiment, the method of any preceding embodiments is disclosed, wherein the step of extracting a plurality of tiles can comprise obtaining at least a first plurality of tiles associated with a first magnification level and a second plurality of tiles associated with a second magnification level, wherein the tiling parameters associated with the extracted tiles comprise the magnification level used to extract the tiles.
5. In an embodiment, the method of embodiments 2-4 is disclosed, wherein magnification levels comprise 1×, 2×, 5×, 10×, 20×.
6. In an embodiment, the method of any preceding embodiments is disclosed, wherein the step of obtaining one or more masks associated with the rasterized representation of the extracted tiles comprises analyzing a rasterized representation of the one or more digital pathology images and associating the one or more masks with the corresponding rasterized representation of extracted tiles.
7. In an embodiment, the method of any preceding embodiments is disclosed, wherein the step of obtaining one or more masks associated with the rasterized representation of the extracted tiles comprises analyzing the rasterized representations of the plurality of extracted tiles and associating the one or more masks with the corresponding rasterized representation of extracted tiles.
8. In an embodiment, the method of any preceding embodiments is disclosed, wherein the step of obtaining one or more masks associated with the rasterized representation of the extracted tiles comprises: analyzing a rasterized representation of the one or more digital pathology images and/or analyzing the rasterized representations of the plurality of extracted tiles, and associating the one or more masks with the corresponding rasterized representations of extracted tiles.
9. In an embodiment, the method of any preceding embodiments is disclosed, wherein the step of obtaining one or more masks associated with the rasterized representation of the extracted tiles comprises obtaining a foreground/background mask.
10. In an embodiment, the method of any preceding embodiments is disclosed, wherein the step of obtaining one or more masks associated with the rasterized representation of the extracted tiles comprises obtaining a foreground/background mask, in particular a tissue/background mask.
11. In an embodiment, the method of any preceding embodiments is disclosed, wherein the step of obtaining one or more masks associated with the rasterized representation of the extracted tiles comprises obtaining a foreground/background mask, in particular a tissue/background mask, wherein the masks parameters comprise the percentage of tissue in the tile.
12. In an embodiment, the method of any preceding embodiments is disclosed, wherein the step of obtaining one or more masks associated with the rasterized representation of the extracted tiles comprises obtaining organ identification masks.
13. In an embodiment, the method of any preceding embodiments is disclosed, wherein the step of obtaining one or more masks associated with the rasterized representation of the extracted tiles comprises obtaining organ identification masks, wherein the masks parameters comprise the number and/or type of organs in the tile.
14. In an embodiment, the method of any preceding embodiments is disclosed, wherein the step of storing a single file comprises storing a single container file.
15. In an embodiment, the method of any preceding embodiments is disclosed, wherein the step of storing a single file, wherein the single file comprises randomly-accessible items.
16. In an embodiment, the method of any preceding embodiments is disclosed, wherein the step of storing a single file comprises storing a single container file, wherein the single container file comprises randomly-accessible items.

17. In an embodiment, the method of any preceding embodiments is disclosed, wherein the single file stored for each of the one or more digital pathology images further comprises a reduced-resolution version of each of the one or more digital pathology images.
18. In an embodiment, the method of embodiment 17 is disclosed, further comprising the step of displaying to a user the reduced-resolution version of each of the one or more digital pathology images.
19. In an embodiment, the method of embodiment 17 is disclosed, further comprising the step of displaying to a user the reduced-resolution version of at least one of the one or more digital pathology images.
20. In an embodiment, the method of any preceding embodiments is disclosed, wherein the step of filtering the training dataset using the tiles metadata comprises selecting one or more single files and a plurality of tiles from each of one or more single files from the training dataset that satisfy one or more predetermined criteria that apply to one or more parameters of the tiles metadata.
21. In an embodiment, the method of any preceding embodiments is disclosed, wherein the step of filtering the training dataset using the tiles metadata comprises selecting one or more single files or a plurality of tiles from each of one or more single files from the training dataset that satisfy one or more predetermined criteria that apply to one or more parameters of the tiles metadata.
22. In an embodiment, the method of any preceding embodiments is disclosed, wherein the step of filtering the training dataset using the tiles metadata comprises selecting one or more single files and/or a plurality of tiles from each of one or more single files from the training dataset that satisfy one or more predetermined criteria that apply to one or more parameters of the tiles metadata.
23. In an embodiment, the method of any preceding embodiments is disclosed, wherein the step of training one or more machine-learning models using the filtered training dataset to obtain one or more trained machine-learning models comprises training a single machine-learning model using the filtered training dataset.
24. In an embodiment, the method of any preceding embodiments is disclosed, wherein the step of training one or more machine-learning models using the filtered training dataset to obtain one or more trained machine-learning models comprises training a single machine-learning model using a plurality of subsets of the filtered training dataset.
25. In an embodiment, the method of any preceding embodiments is disclosed, wherein the step of training one or more machine-learning models using the filtered training dataset to obtain one or more trained machine-learning models comprises training multiple machine-learning models using the filtered training dataset.
26. In an embodiment, the method of any preceding embodiments is disclosed, wherein the step of training one or more machine-learning models using the filtered training dataset to obtain one or more trained machine-learning models comprises training multiple machine-learning models using a plurality of subsets of the filtered training dataset.
27. In an embodiment, the method of any preceding embodiments is disclosed, wherein the step of training one or more machine-learning models using the filtered training dataset to obtain one or more trained machine-learning models comprises training a single machine-learning model using the filtered training dataset, training a single machine-learning model using a plurality of subsets of the filtered training dataset, training multiple machine-learning models using the filtered training dataset, training multiple machine-learning models using a plurality of subsets of the filtered training dataset.
28. In an embodiment, a computer-implemented method of selecting one or more machine-learning models for digital pathology image analysis from one or more trained machine-learning models obtained according to any of the preceding embodiments is disclosed, the method comprising the steps of:
   a. receiving a test dataset comprising at least one digital pathology image;
   b. testing the one or more trained machine-learning models using the test dataset;
   c. calculating one or more evaluation metrics for each of the one or more tested machine-learning models;
   d. selecting one or more tested machine-learning models using one or more predetermined criteria applying to the one or more evaluation metrics.
29. In an embodiment, a computer-implemented method of using a trained machine-learning model, obtained according to any of the preceding embodiments, to analyze digital pathology images is disclosed, the method comprising the steps of:
   a. receiving at least one digital pathology image;
   b. extracting, via the trained machine-learning model, features of the received at least one digital pathology image, wherein extracting features comprises detecting and/or predicting the presence of objects, structures, lesions in the received at least one digital pathology image.
30. In an embodiment, a computer-implemented method of using a trained machine-learning model, obtained according to any of the preceding embodiments, to analyze digital pathology images is disclosed, the method comprising the steps of:
   a. receiving at least one digital pathology image;
   b. extracting, via the trained machine-learning model, features of the received at least one digital pathology image, wherein extracting features comprises detecting and predicting the presence of objects, structures, lesions in the received at least one digital pathology image.
31. In an embodiment, a computer-implemented method of using a trained machine-learning model, obtained according to any of the preceding embodiments, to analyze digital pathology images is disclosed, the method comprising the steps of:
   a. receiving at least one digital pathology image;
   b. extracting, via the trained machine-learning model, features of the received at least one digital pathology image, wherein extracting features comprises detecting or predicting the presence of objects, structures, lesions in the received at least one digital pathology image.
32. In an embodiment, a system is disclosed, the system comprising:
   a. a processor; and
   b. a computer readable medium comprising instructions that, when executed by the processor, cause the processor to perform the steps of the method of any preceding embodiments.
33. In an embodiment, a system is disclosed, the system comprising:
   a. a processor; and
   b. a computer readable medium comprising instructions that, when executed by the processor, cause the processor to perform the steps of the method of any preceding embodiments; and
   c. optionally a digital pathology image acquisition means.

1. A computer-implemented method of obtaining one or more trained machine-learning models for digital pathology image analysis, the method comprising the steps of:
a. receiving one or more digital pathology images (20), optionally comprising receiving annotations associated to the one or more digital pathology images (20A);
b. preprocessing the one or more digital pathology images (22), wherein preprocessing comprises, for each of the one or more digital pathology images:
i. extracting a plurality of tiles (22A);
ii. obtaining a rasterized representation of the extracted tiles (22B);
iii. obtaining one or more masks associated with the rasterized representation of the extracted tiles (22C);
iv. associating, with each tile, tile metadata comprising one or more tiling parameters, one or more masks parameters, and optionally the received annotations associated to the digital pathology image from which the tile is extracted (22D);
v. storing a single file, the single file comprising the rasterized tiles, the obtained masks and the tiles metadata (22E);
c. obtaining a training dataset comprising the one or more single files stored for the one or more preprocessed digital pathology images (24);
d. filtering the training dataset using the tiles metadata (26);
e. training the one or more machine-learning models using the filtered training dataset to obtain one or more trained machine-learning models (28);
f. outputting the one or more trained machine-learning models (30).

2. The method of claim 1, wherein the step of extracting a plurality of tiles can comprise obtaining at least a first plurality of tiles associated with a first magnification level and a second plurality of tiles associated with a second magnification level, wherein the tiling parameters associated with the extracted tiles comprise the magnification level used to extract the tiles.

3. The method of claim 2, wherein magnification levels comprise 1×, 2×, 5×, 10×, 20×.

4. The method of claim 1, wherein the step of obtaining one or more masks associated with the rasterized representation of the extracted tiles comprises: analyzing a rasterized representation of the one or more digital pathology images and/or analyzing the rasterized representations of the plurality of extracted tiles, and associating the one or more masks with the corresponding rasterized representations of extracted tiles.

5. The method of claim 1, wherein the step of obtaining one or more masks associated with the rasterized representation of the extracted tiles comprises obtaining a foreground/background mask, in particular a tissue/background mask, wherein the masks parameters comprise the percentage of tissue in the tile.

6. The method of claim 1, wherein the step of obtaining one or more masks associated with the rasterized representation of the extracted tiles comprises obtaining organ identification masks, wherein the masks parameters comprise the number and/or type of organs in the tile.

7. The method of claim 1, wherein the step of storing a single file comprises storing a single container file, wherein the single container file comprises randomly-accessible items.

8. The method of claim 1, wherein the single file stored for each of the one or more digital pathology images further comprises a reduced-resolution version of each of the one or more digital pathology images.

9. The method of claim 8, further comprising the step of displaying to a user the reduced-resolution version of each of the one or more digital pathology images.

10. The method of claim 1, wherein the step of filtering the training dataset using the tiles metadata comprises selecting one or more single files and/or a plurality of tiles from each of one or more single files from the training dataset that satisfy one or more predetermined criteria that apply to one or more parameters of the tiles metadata.

11. The method of claim 1, wherein the step of training one or more machine-learning models using the filtered training dataset to obtain one or more trained machine-learning models comprises training a single machine-learning model using the filtered training dataset, training a single machine-learning model using a plurality of subsets of the filtered training dataset, training multiple machine-learning models using the filtered training dataset, training multiple machine-learning models using a plurality of subsets of the filtered training dataset.

12. A computer-implemented method of selecting one or more machine-learning models for digital pathology image analysis from one or more trained machine-learning models obtained according to claim 1, the method comprising the steps of:
a. receiving a test dataset comprising at least one digital pathology image (50);
b. testing the one or more trained machine-learning models using the test dataset (52);
c. calculating one or more evaluation metrics for each of the one or more tested machine-learning models (54);
d. selecting one or more tested machine-learning models using one or more predetermined criteria applying to the one or more evaluation metrics (56).

13. A computer-implemented method of using a trained machine-learning model, obtained according to claim 1, to analyze digital pathology images, the method comprising the steps of:
a. receiving at least one digital pathology image (60);
b. extracting, via the trained machine-learning model, features of the received at least one digital pathology image, wherein extracting features comprises detecting and/or predicting the presence of objects, structures, lesions in the received at least one digital pathology image (62).

14. A system comprising:
a. a processor; and
b. a computer readable medium comprising instructions that, when executed by the processor, cause the processor to perform the steps of the method of claim 1;
c. a digital pathology image acquisition means.

15. (canceled)
